(19) United States
(12) Patent Application Publication
(10) Pub. No.: US 2017/0190645 A1
(43) Pub. Date: Jul. 6, 2017

(54) APPARATUS FOR PRODUCING CHEMICAL PRODUCT AND METHOD FOR PRODUCING CHEMICAL PRODUCT

(71) Applicant: SEKISUI CHEMICAL CO., LTD., Osaka (JP)

(72) Inventor: Satoshi KOMA, Tokyo (JP)

(73) Assignee: SEKISUI CHEMICAL CO., LTD., Osaka (JP)

(21) Appl. No.: 15/325,162

(22) PCT Filed: Jul. 24, 2015
(86) PCT No.: PCT/JP2015/071140

§ 371 Date: Jan. 10, 2017

(30) Foreign Application Priority Data
Jul. 30, 2014 (JP) 2014-155501

(51) Int. Cl. C07C 29/152; B01J 19/24; B01J 7/00; C01B 3/02; C01B 3/34

(52) U.S. Cl. CPC C07C 29/152; C01B 3/02; C01B 3/34; B01J 7/00; B01J 19/245; C01B 2203/0244; C01B 2203/061; C01B 2203/0838; C01B 2203/1241; C01B 2203/148; B01J 2219/24

(57) ABSTRACT

The present invention provides an apparatus and a method which can be used for improving energy efficiency in production of a chemical product. The apparatus 1 for producing a chemical product comprises a synthesis gas generation unit 11, a synthesis unit 16, and a reforming unit 12. The synthesis gas generation unit 11 generates a synthesis gas by partial oxidation of these carbon sources. The synthesis unit 16 subjects the provided synthesis gas to catalytic reaction so as to synthesize a chemical product. The reforming unit 1 reforms hydrocarbons generated in at least one of the synthesis gas generation unit 11 and the synthesis unit 16 into carbon monoxide and hydrogen, and feeds them into the synthesis unit 16. The reforming unit 12 reforms the hydrocarbons by utilizing heat exhausted from the synthesis gas generation unit 11.

### TECHNICAL FIELD

The present invention relates to an apparatus and a method for producing a chemical product.

### DESCRIPTION OF RELATED ART

Conventionally, a method is known in which ethanol is produced by reacting a synthesis gas obtained from biomass in the presence of a catalyst (see Patent Document 1). The catalytic reaction of a synthesis gas may result in generation of by-product such as methane, ethane and ethylene, in addition to ethanol as a target product. Patent Document 1 describes the reformation of such by-products into carbon monoxide and hydrogen.

### DOCUMENTS OF RELATED ART

### Patent Document

[Patent Document 1] Japanese Unexamined Patent Application Publication No. 2012-149089

### SUMMARY OF THE INVENTION

### Problems to be Solved by the Invention

It has been desired to improve energy efficiency in production of a chemical product such as bioethanol.

It is a primary object of the present invention to provide an apparatus and a method which can be used for improving energy efficiency in production of a chemical product such as an alcohol.

### Means to Solve the Problems

With respect to the apparatus of the present invention for producing a chemical product, the apparatus comprises a synthesis gas generation unit, a synthesis unit and a reforming unit. The synthesis gas generation unit generates a synthesis gas by partial oxidation of a carbon source. The synthesis unit synthesizes a chemical product by subjecting the synthesis gas to catalytic reaction. The reforming unit reforms hydrocarbons generated in at least one of the synthesis gas generation unit and the synthesis unit into carbon monoxide and hydrogen, and feeds the carbon monoxide and the hydrogen into the synthesis unit. The reforming unit is configured to reform the hydrocarbons by utilizing heat exhausted from the synthesis gas generation unit.

In the apparatus of the present invention, the reforming unit may reform hydrocarbons which are generated in and exhausted from both of the synthesis gas generation unit and the synthesis unit.

In the apparatus of the present invention, the reforming unit may be configured to reform the hydrocarbons having 1 to 6 carbon atoms.

In the apparatus of the present invention, the reforming unit may be configured to reform methane.

### DETAILED DESCRIPTION OF THE INVENTION

With respect to the method of the present invention for producing a chemical product, the method comprises: a step of performing partial oxidation of a carbon source to generate a synthesis gas; a step of performing catalytic reaction of the synthesis gas in a synthesis unit to synthesize a chemical product; and a step of reforming hydrocarbons generated in at least one of the step of performing partial oxidation of the carbon source and the step to synthesize the chemical product into carbon monoxide and hydrogen, and feeding the carbon monoxide and the hydrogen into the synthesis unit. Further, the reforming of the hydrocarbons is performed by utilizing heat exhausted from the step of performing partial oxidation of the carbon source.

### EFFECT OF THE INVENTION

As described above, the present invention can provide an apparatus and a method which can be used for improving energy efficiency in production of a chemical product.

### BRIEF DESCRIPTION OF THE DRAWINGS

FIG. 1 is a schematic view of the apparatus for producing a chemical product according to a first embodiment of the present invention.

FIG. 2 is a schematic view of the apparatus for producing a chemical product according to a second embodiment of the present invention.

### DESCRIPTION OF THE EMBODIMENTS

Hereinbelow, the present invention will be described in detail with reference to preferred embodiments of the present invention. However, these embodiments are only examples. The present invention is in no way limited by these embodiments.

### First Embodiment

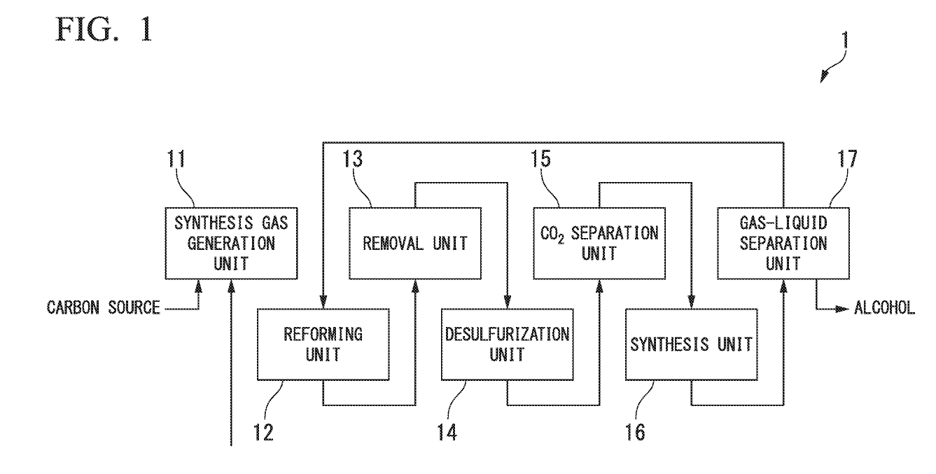

FIG. 1 is a schematic view of the apparatus for producing a chemical product according to a first embodiment of the present invention. The apparatus 1 shown in FIG. 1 is an apparatus for producing a chemical product from organic waste, biomass and the like.

The apparatus 1 comprises a synthesis gas generation unit 11. A non-fossil organic resource (i.e., carbon source) such as organic wastes, biomass or the like is fed into the synthesis gas generation unit 11. The synthesis gas generation unit 11 generates a synthesis gas by partial oxidation of these carbon sources. Generally, the partial oxidation of a carbon source results in generation of by-products such as hydrocarbons, and carbon dioxide. Therefore, the synthesis gas obtained in the synthesis gas generation unit 11 usually contains hydrocarbons, carbon dioxide, and the like, in addition to carbon monoxide and hydrogen. Subsequently, the synthesis gas generation unit 11 feeds the obtained synthesis gas into a reforming unit 12. The hydrocarbons contained in the synthesis gas as by-products in the synthesis gas generation unit 11 include hydrocarbons having 1 to 6 carbon atoms. Specifically, the hydrocarbons include a large amount of methane.

The reforming unit 12 reforms the hydrocarbons contained in the synthesis gas into carbon monoxide and hydrogen. Subsequently, the reforming unit 12 feeds the reformed synthesis gas into a removal unit 13.

The removal unit 13 removes impurities contained in the synthesis gas. Specifically, in the removal unit 13, the impurities contained in the synthesis gas are dissolved in a solution or the like so as to remove the impurities from the synthesis gas. Subsequently, the resulting synthesis gas free of impurities is fed from the removal unit 13 into a desulfurization unit 14.

The desulfurization unit 14 separates sulfur content off from the synthesis gas so as to obtain a desulfurized synthesis gas. Subsequently, the desulfurization unit 14 feeds the desulfurized synthesis gas into a carbon dioxide separation unit 15.

The carbon dioxide separation unit 15 separates carbon dioxide off from the synthesis gas so as to obtain a carbon dioxide-free synthesis gas. Subsequently, the carbon dioxide separation unit 15 feeds the carbon dioxide-free synthesis gas into a synthesis unit 16.

The synthesis unit 16 subjects the synthesis gas to catalytic reaction so as to synthesize a chemical product. Specifically, the synthesis unit 16 synthesizes a chemical product such as an alcohol and the like by catalytic reaction of carbon monoxide and hydrogen, which are contained in the resultant synthesis gas. As an example of a catalyst used in the catalytic reaction, a four-way catalyst composed of rhodium, manganese, lithium and magnesium, and the like, can be mentioned. Generally, the catalytic reaction of the synthesis gas results in generation of by-products such as hydrocarbons, Accordingly, the synthesis unit 16 feeds the resultant components including the target chemical product such as an alcohol as well as by-products into the gas-liquid separation unit 17.

The gas-liquid separation unit 17 separates the components into gas and liquid. Consequently, in the gas-liquid separation unit 17, the liquid components including a chemical product such as an alcohol can be obtained. The gas-liquid separation unit 17 may be connected to a purification unit for purifying a chemical product such as an alcohol from the liquid components.

In the gas-liquid separation unit 17, the separated gas includes unreacted carbon monoxide and hydrogen, and hydrocarbons and the like. The gas-liquid separation unit 17 feeds these gas components to the reforming unit 12.

The reforming unit 12 reforms hydrocarbons contained in the separated gas into carbon monoxide and hydrogen. Therefore, the carbon monoxide and hydrogen, which are obtained by the reforming of the hydrocarbons contained in the separated gas, and the unreacted carbon monoxide and hydrogen contained also in the separated gas, are fed to the synthesizing unit 16 and used for synthesizing a chemical product such as an alcohol.

The reforming reaction performed in the reforming unit 12 is an endothermic reaction. Accordingly, it is necessary to supply heat to the reforming unit. In the apparatus 1, the heat exhausted from the synthesis gas generation unit 11 is supplied to the reforming unit 12. The reforming unit 12 reforms the hydrocarbons by utilizing heat exhausted from the synthesis gas generation unit 11. Consequently, the amount of external heat to be supplied to the reforming unit 12 can be reduced. Thus, the first embodiment of the present invention can provide an apparatus and a method which can be used for improving energy efficiency production of a chemical product.

Hereinbelow, the present invention will be described in detail with reference to further preferred embodiments of the present invention. In the following descriptions, components having substantially the same functions as those in the first embodiment are denoted with the same reference numerals, and the descriptions thereof are omitted.

### Second Embodiments

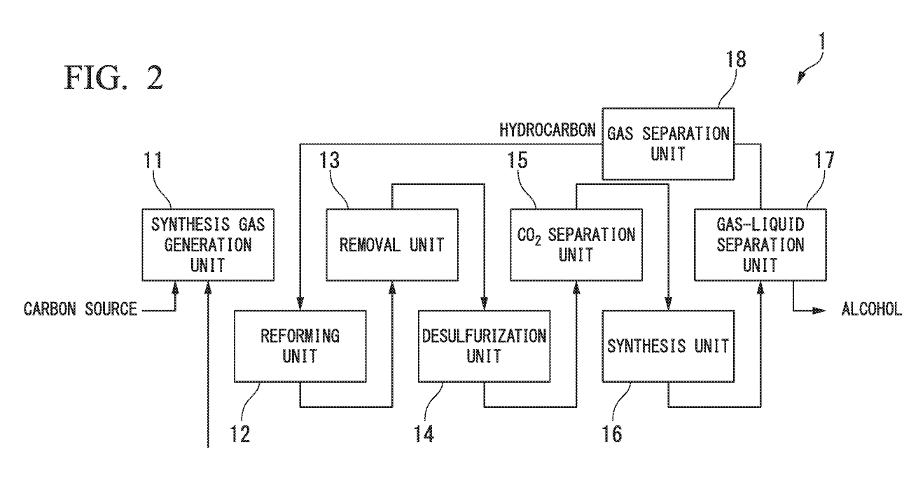

FIG. 2 is a schematic view of the apparatus for producing a chemical product according to a second embodiment of the present invention. In the second embodiment, a gas separation unit 18 is connected to the gas-liquid separation unit 17. The gas separated in the gas-liquid separation unit 17 is fed into the gas separation unit 18. The gas separation unit 18 separates the gas into hydrocarbons and other components. The gas separation unit 18 feeds the obtained hydrocarbons into the reforming unit 12. The gas separation unit 18 feeds the obtained other components including unreacted carbon monoxide and hydrogen into the synthesis unit 16. Thus, this embodiment of the present invention makes it possible to feed only the hydrocarbons separately from other gas components separated by the gas-liquid separation unit 17 into the reforming unit 12. Accordingly, the reforming of the hydrocarbons can be performed more efficiently.

### DESCRIPTION OF THE REFERENCE SIGNS

1: Apparatus
11: Synthesis gas generation unit
12: Reforming unit
13: Removal unit
14: Desulfurization unit
15: Carbon dioxide separation unit
16: Synthesis unit
17: Gas-liquid separation unit
18: Gas separation unit

1. An apparatus for producing a chemical product, comprising:
a synthesis gas generation unit for generating a synthesis gas by partial oxidation of a carbon source;
a synthesis unit for synthesizing a chemical product by subjecting the synthesis gas to catalytic reaction; and
a reforming unit for reforming hydrocarbons generated in at least one of the synthesis gas generation unit and the synthesis unit into carbon monoxide and hydrogen, and feeding the carbon monoxide and the hydrogen into the synthesis unit,
the reforming unit being configured to reform the hydrocarbons by utilizing heat exhausted from the synthesis gas generation unit.

2. The apparatus according to claim 1, wherein the reforming unit reforms the hydrocarbons which are generated in and exhausted from both of the synthesis gas generation unit and the synthesis unit.

3. The apparatus according to claim 1, wherein the reforming unit is configured to reform the hydrocarbons having 1 to 6 carbon atoms.

4. The apparatus according to claim 3, wherein the reforming unit is configured to reform methane.

5. A method for producing a chemical product, comprising:
a step of performing partial oxidation of a carbon source to generate a synthesis gas;
a step of synthesizing a chemical product by subjecting the synthesis gas to catalytic reaction in a synthesis unit,
a step of reforming hydrocarbons generated in at least one of the step of performing partial oxidation of the carbon source and the step of synthesizing the chemical product into carbon monoxide and hydrogen, and feeding the carbon monoxide and the hydrogen into the synthesis unit,
the reforming of the hydrocarbons being performed by utilizing heat exhausted from the step of performing partial oxidation of the carbon source.

6. The apparatus according to claim 2, wherein the reforming unit is configured to reform the hydrocarbons having 1 to 6 carbon atoms.

7. The apparatus according to claim 6, wherein the reforming unit is configured to reform methane.
